(19) United States
(12) Patent Application Publication
(10) Pub. No.: US 2024/0033314 A1
(43) Pub. Date: Feb. 1, 2024

(54) COMPOSITION CONTAINING AN OSMOTIC SUBSTANCE, AND USE OF THE COMPOSITION

(71) Applicants: NOVIGO GMBH, Grünberg (DE); Sidroga AG, Rheinfelden (CH)

(72) Inventor: Thomas Schmidts, Giessen (DE)

(21) Appl. No.: 18/258,098

(22) PCT Filed: Dec. 16, 2021
(86) PCT No.: PCT/EP2021/086327

§ 371 Date: Jun. 16, 2023

(30) Foreign Application Priority Data
Dec. 16, 2020 (EP) 20214780.7

(51) Int. Cl. A61K 36/886; A61K 9/06; A61K 35/02; A61K 9/00; A61K 47/38; A61P 29/00; A61P 17/00; A61K 36/68

(52) U.S. Cl. CPC A61K 36/886; A61K 9/06; A61K 35/02; A61K 9/0014; A61K 47/38; A61P 29/00; A61P 17/00; A61K 36/68

(57) ABSTRACT

The invention relates to a composition for use in the treatment of skin irritations due to interactions with plants or animals on or in the skin, the composition containing at least one osmotic and/or hygroscopic substance.

### CROSS-REFERENCE TO RELATED APPLICATIONS

This application is a filing under 35 U.S.C. 371 of International Application No. PCT/EP2021/086327, filed Dec. 16, 2021, entitled “Composition Containing an Osmotic Substance, and Use of the Composition,” which claims priority to EP Patent Application No. 20214780.7, filed Dec. 16, 2020, which applications are incorporated by reference herein in their entirety.

### FIELD OF THE DISCLOSURE

The present disclosure generally relates to a composition for treating skin irritations caused by plant or animal effects on or in the skin, the composition containing at least one osmotically and/or hygroscopically active substance.

### BACKGROUND

Skin irritations caused by animal or plant effects can currently mostly be treated only with little satisfaction. Animal effects include, for example, stings and bites by arthropods, in particular insects and arachnids, and the application of sting toxins, for example by caterpillars of the processionary moth, in particular caterpillars of the oak processionary moth, and cnidarians, in particular jellyfish. Alternatively, sting toxins can be applied onto or into the skin also through the action of stinging nettles and therefore be of plant origin. Furthermore, by way of skin contact with thorns, spines, needles, hairs and other forms of emergences and trichomes, plants can cause injuries and irritations of the skin, such as giant hogweed, conifers and firs.

Skin irritations include, for example, redness, swelling, wheals and itching in the region of the affected site.

Insect bites and stings include in particular bites or stings by hymenopterans (Hymenoptera), dipterans (Diptera) and neopterans (Neoptera). Hymenopterans (Hymenoptera) include, for example, bees, wasps and ants. Dipterans include, for example, mosquitoes and horse flies. Neopterans include, for example, fleas, lice and bugs.

Arachnids (Arachnida) include not only spiders but also mites and ticks.

Currently, on the one hand, there are preparations for treating the skin irritation which alleviate the symptoms, in particular the itching, but do not help to eliminate the cause. These include, for example, preparations that alleviate the itching only by cooling or by treatment with pharmacologically active substances, such as cortisone derivatives or antihistamines. However, these preparations do not eliminate the cause of the skin irritation, particularly the secretion secreted by animals or plants or the sting toxin of plant or animal origin.

On the other hand, there are treatment methods that help to eliminate the cause of the skin irritation; however, they are expensive and uncomfortable, or even painful. These treatment methods include, for example, the treatment of a puncture site with heat or reduced pressure. Heating of the puncture site is intended to denature the secretion, particularly the secretion secreted by mosquitoes that is in the form of irritants and/or toxin, this being intended to prevent itching. However, this treatment is painful at least briefly. Treatment of the skin irritation with reduced pressure, for example by using vacuum pumps, is intended to remove the secretion through the puncture channel or bite site. However, this is expensive and prone to error in the handling and leads to damage to the skin as a result of the reduced pressure.

### SUMMARY

It is therefore the object of the disclosure to eliminate the disadvantages of the prior art and to provide a composition which can be applied topically, alleviates skin irritations caused by plant and animal effects, in particular by stings and bites by insects and spiders and by sting toxins, and reduces, or even removes, the cause of the skin irritation.

Disclosed is a composition for use in the treatment of skin irritations caused by plant or animal effects on or in the skin, the composition containing at least one osmotically and/or hygroscopically active substance. Also disclosed is a method for applying the composition topically to a skin of an animal or a human.

Other technical features may be readily apparent to one skilled in the art from the following FIGURES, descriptions and claims.

### BRIEF DESCRIPTION OF THE DRAWINGS

For a more complete understanding of this disclosure, reference is now made to the following description, taken in conjunction with the accompanying drawings, in which:

FIG. 1. shows an exploded drawing of a Franz diffusion chamber.

### DESCRIPTION

Plant and animal effects on or in the skin include in particular the application of secretion by arthropods, for example by insect stings or spider bites, and of sting toxin, for example from plant sources or of animal origin, in particular by stinging nettles, jellyfish or caterpillars of the processionary moth. Application is effected for example by way of injury to the skin by stinging hairs or by puncture or bite sites, where the foreign substance, in particular secretion or toxin, which triggers the respective skin irritation at least partially penetrates into the skin.

In this document, “secretion” is used for any at least partially liquid or dissolved foreign substance of animal or plant origin, for example saliva, toxins or other irritants. Secretions that are applied into the skin for example in the case of an insect sting have an irritating and skin-irritating effect and are potentially inflammatory.

Osmotically active substances are substances for which a solvent-permeable membrane is impermeable. A difference in concentration between two compartments separated by a membrane, on account of osmotically active substances, can therefore be compensated for only by a liquid flow.

Hygroscopically active substances attract moisture from the environment and therefore have a similar suction effect on liquids as osmotically active substances.

This effect is used to treat skin irritations caused by animal and plant effects. The foreign substance applied into the skin by animals or plants which causes the skin irritation and cannot be easily removed mechanically, for example by wiping, rinsing the skin or pulling out a spine, is removed from the skin by means of the osmotically and/or hygroscopically active substance in the topically applied composition. In particular, the foreign substance introduced into the skin, which may for example be a secretion or sting toxin, is drawn out of the puncture or bite site and thus removed. This treats the cause of the skin irritation painlessly and without damaging the skin.

The topical application of a composition for treating skin irritations due to animal or plant effects having an osmotically and/or hygroscopically active substance thus enables the gentle removal of a foreign substance, causing the skin irritation, from the skin based on the difference in concentration between the foreign substance and the composition. This composition can have a concentration of osmotically and/or hygroscopically active substance greater than the concentration in the foreign substance, in particular secretion or toxin, or the physiological concentration of the skin.

A composition here is a combination of multiple substances and ingredients that together form a preparation that can be applied to skin, particularly in the area of a skin irritation, of a sting site or bite site, for topical treatment.

The skin exhibiting a skin irritation is in particular of human and/or animal origin. The composition is thus suitable for treating skin irritations, in particular caused by stings or bites by arthropods or else plant or animal sting toxins, in humans and/or animals.

In aspects, at least one osmotically and/or hygroscopically active substance in the composition is a salt or a low molecular weight organic substance, for example, a sugar.

This makes it possible to obtain a composition for application to the skin in the area of a skin irritation which reduces, or even completely eliminates, the causes of the skin irritation and at the same time is well tolerated by the skin, without having any negative side effects.

In an embodiment, the composition contains multiple osmotically and/or hygroscopically active substances. If multiple osmotically and/or hygroscopically active substances are present in the composition, for example—two or more different salts—or—two or more different sugars—or—one or more salts in combination with one or more sugars—may be present. The use of multiple salts or sugars in the composition makes it possible to cumulatively exploit further advantageous properties in addition to the osmotic and/or hygroscopic activity. For example, in addition to highly osmotically and/or hygroscopically active salts, use may additionally be made of salts that have an additional anti-inflammatory effect.

In an embodiment, the osmotically and/or hygroscopically active substance comprises or consists of a non-toxic inorganic or organic salt. In particular, an inorganic salt, as the osmotically and/or hygroscopically substance, has a cation selected from the group: sodium, potassium, lithium, magnesium, and/or calcium, and an anion selected from the group of sulfate, sulfide, chloride, bromide, iodide, fluoride or carbonate. An organic salt of the osmotically active substance and/or hygroscopically active substance in particular has a cation selected from the group of quaternary ammonium compounds or organically substituted ammonium compounds and an anion selected from the group of carboxylates or organic sulfates. Alternatively, the osmotically and/or hygroscopically active substance comprises or consists of a salt from the group of amino acids or betaines.

In an alternative or additional embodiment, the osmotically and/or hygroscopically active substance comprises or consists of a sugar, for example, one or more mono- and/or disaccharides, sugar alcohols, in particular mannitol, sorbitol, xylitol and/or isomalt.

In aspects, the composition contains a mineral salt as the osmotically and hygroscopically active substance. “Mineral salt” denotes a composition consisting of multiple inorganic compounds and salts. This includes, for example, mineral salt from thermal springs in the town of Bad Ems, which is also commercially available under the name Emser Salt®. In addition to sodium chloride and hydrogen carbonate, Emser Salt® has more than 30 mineral substances and trace elements.

According to analysis, Emser salt as mineral salt comprises, per kg of mineral salt, 308.7 g of sodium ions, 6.11 g of potassium ions, 0.291 g of magnesium ions, 0.21 g of lithium ions, 0.016 g of calcium ions, 0.003 g of iron(II, III) ions, 0.0001 g of manganese ions, 474.4 g of hydrogen carbonate ions, 188.4 g of chloride ions, 14.0 g of carbonate ions, 9.24 g of sulfate ions, 0.355 g of nitrate ions, 0.202 g of bromide ions, 0.078 g of fluoride ions and 0.005 g of iodide ions.

In aspects, the composition contains at least one osmotically and/or hygroscopically active substance at a concentration of 1% to 70% by weight; alternatively, at a concentration of 1% to 20% by weight; alternatively, of 1.5% to 2.5% by weight of the composition.

For example, the composition contains 1% to 30% of salt or 1% to 70% of sugar as the osmotically and/or hygroscopically active substance.

In the case of mineral salt, very good efficacy was in particular found with at least 2% by weight of mineral salt.

The use of the stated proportions by weight of osmotically and/or hygroscopically active substance, in particular in the range from 1.5% to 30% by weight in the form of a salt, makes it possible to achieve a sufficient effect on the skin irritation, without the osmotically active substance displaying negative properties, for example crystallization, drying-out of the skin or a sticky feel on the skin.

The osmotic and/or hygroscopic substance can be in dissolved or dispersed form in the composition. This makes it possible to achieve sufficient efficacy of the substance when applied topically onto the skin.

The composition can contain at least one solvent, in particular water as solvent. Alternatively or additionally, the composition contains ethanol as solvent, which at the same time has a disinfecting effect. Further examples of solvents are other mono- or polyhydric alcohols and polyols, for example propylene glycol, pentylene glycol, butylene glycol. The composition can contain one or more different solvents. As a result, different polarities and thus solution effects can be exploited and in particular adapted to the osmotically and/or hygroscopically active substance.

In addition, the solvent or multiple different solvents can be selected in such a way that lipophilic foreign substances and/or toxins can be better extracted from the skin. For this, the polarity of the composition can be adapted to the field of application or to the foreign substance and/or the toxin.

In an embodiment, the composition is in the form of a gel. For this, the composition contains at least one gel former. This type of formulation enables the use of the osmotic and/or hygroscopic substance in dispersed form.

Gels have the property of having a cooling effect at the same time as an effect of a contained active ingredient.

In addition, the composition in gel form can be applied to the skin in the area of the skin irritation, in particular in the area of the skin irritation.

The composition can contain a carbomer and/or a cellulose derivative as gel former and thickener, for example hydroxyethylcellulose. Hydroxyethylcellulose is available as a semisynthetic gel former for example under the trade name NATROSOL™ 250 HX.

In alternative embodiments, the composition may be in the form of a cream, emulsion or of a preparation for a stick, roll-on, patch or a spray. These various application forms enable the composition to be applied or sprayed onto the skin and ensure that the composition remains on the skin for a sufficiently long time for a foreign substance, for example a secretion or sting toxin, to be able to be drawn out of the skin. This is based on the suction effect of the osmotically and/or hygroscopically active substance particularly caused by a concentration gradient.

In an embodiment, the composition has at least one additive that has an effect on the skin irritation. The at least one additive can be selected from the group of humectants, plant extracts or buffers. This makes it possible to achieve further advantageous effects on the skin irritation and the surrounding skin.

The humectant is glycerin and/or ethylhexylglycerin, for example. Plant extracts may be, for example, aloe vera extract, sage extract or ribwort plantain extract. For example, lactic acid may be added as a buffer and for pH regulation.

The composition may also contain further active ingredients, extracts with supportive properties against redness, inflammation and/or with an antibacterial effect, or else substances of non-plant origin with the stated properties, for example cortisone. An active ingredient with a cooling property is also betaine, for example.

A cooling effect can be obtained through the use of aloe vera extracts, glycerin and/or ethanol. In addition, aloe vera extracts and glycerin provide moisture to the irritated skin and therefore care for it. These ingredients may also be used in another application form, whereby a cooling effect is achieved independently of gel properties of the composition.

In an embodiment, the composition contains at least the following ingredients: 80%-99% by weight of solvent and 1%-20% by weight; alternatively, 1.5% to 2.5% by weight; alternatively, 2% by weight, of osmotically and/or hygroscopically active substance.

The solvent here may be formed for example by 0.1%-60% by weight of ethanol and the remaining amount of water.

In aspects, the composition in gel form contains at least the following ingredients: 80%-99% by weight of solvent; 1%-20% by weight, alternatively 1.5% to 2.5% by weight, alternatively 2% by weight, of osmotically and/or hygroscopically active substance; and 0.1%-5% by weight, alternatively 1%-2% by weight, alternatively 1.5% by weight, of gel former.

In aspects, the composition in gel form having additives contains at least the following ingredients: 80%-99% by weight of solvent; 1%-20% by weight, alternatively 1.5% to 2.5% by weight, alternatively 2% by weight, of osmotically and/or hygroscopically active substance, 0.1%-5% by weight, alternatively 1%-2% by weight, alternatively 1.5% by weight, of gel former and 0.0001%-20% by weight of additives.

In aspects, the composition in gel form having additives contains at least the following ingredients:
80%-99% by weight of solvent,
1%-20% by weight, alternatively 1.5% to 2.5% by weight, alternatively 2% by weight, of osmotically and/or hygroscopically active substance,
0.1%-5% by weight, alternatively 1%-2% by weight, alternatively 1.5% by weight, of gel former and
0.0001%-20% by weight of additives, in particular:
   humectants 0.1%-20% by weight, alternatively 1%-5% by weight, alternatively 3% by weight,
   plant extracts 0.0001%-20% by weight, alternatively 0.01% by weight to 1.5% by weight, and
   buffer as required, depending on the pH of the composition 0.1%-5% by weight.

In aspects, the composition consists of the following ingredients:
80%-99% by weight of solvent, in particular:
   ethanol 0.1%-60% by weight, alternatively 20% based on a total weight of the solvent,
   water 20%-99% by weight, to make up to 100% of the solvent,
1%-20% by weight, alternatively 1.5% to 2.5% by weight, alternatively 2% by weight, of osmotically and/or hygroscopically active substance (e.g., a mineral salt),
0.1%-5% by weight, alternatively 1%-2% by weight, alternatively 1.5% by weight, of gel former (e.g., hydroxyethylcellulose), and
0.0001%-20% by weight of additives, in particular:
   humectants 0.1%-20% by weight, alternatively 1%-5% by weight, alternatively 3% by weight (e.g., glycerin as humectant),
   plant extracts 0.0001%-20% by weight, alternatively 0.01% by weight to 1.5% by weight, for example:
      ribwort plantain extract at 0.1%-20% by weight, alternatively 1% by weight, and
      aloe vera extract at 0.001%-5% by weight, alternatively 0.03% by weight,
   a buffer as required (e.g., lactic acid), depending on the pH of the composition 0.1%-5% by weight.

In addition, the invention relates to the use of a composition having at least one osmotically active substance for the treatment of skin irritations caused by plant or animal effects on or in the skin.

The composition can be applied directly after an insect sting, spider bite or contact with sting toxin has occurred. Unexpectedly, the composition works even when it is not applied immediately, but also after several hours and once severe swelling has already occurred.

In an embodiment, the composition is applied as a gel. This makes it possible to apply the active ingredient in the form of the osmotically and/or hygroscopically active substance in a controlled and sustained manner.

### Investigations of the Active Ingredient Properties—Evidence of the Osmotic Activity by Means of the Franz Diffusion Chamber

The aim of the experiment was to demonstrate that the composition is suitable for extracting foreign substances, which cause skin irritations, from the skin by means of an osmotically active substance.

For this, a composition in the form of a gel having 2% by weight of salt as osmotically active substance, dissolved in water as solvent and admixed with hydroxyethylcellulose as gel former, was tested against a physiological saline solution (0.9% saline solution). The exchange is effected via a semi-permeable membrane with a tiny puncture hole in order to simulate the skin with a puncture or bite site. The salt used was mineral salt from a thermal spring in the town of Bad Ems, in particular Emser® salt.

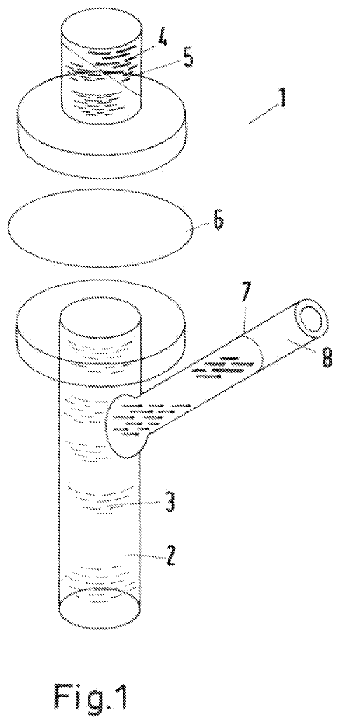

The effect of the composition, that of removing foreign substances, in the form of secretions or toxins, from the skin by means of an osmotically active substance and a concentration gradient, was demonstrated by means of a Franz diffusion chamber 1. FIG. 1 shows an exploded drawing of such a Franz diffusion chamber 1. As reference composition 2, a 0.9% saline solution, stained with the membrane-permeable dye fluorescein sodium (C.I.45350) for better visibility, was added into the acceptor chamber 3 in order to model the blood.

The donor chamber 4, into which the test composition 5 had been added, was connected to the acceptor chamber 3 via a membrane 6. A tiny hole was pierced into the membrane using a cannula in order to simulate the sting channel.

A composition in gel form having 2% by weight of salt as osmotically and hygroscopically active substance, dissolved in water as solvent and hydroxyethylcellulose as gel former, was used as test composition 5.

As a control, in a separate Franz diffusion chamber 1, the acceptor chamber 3 was also filled with 0.9% saline solution and membrane-permeable dye, and a non-inventive composition without osmotically or hygroscopically active substance and gel former as test composition 5 was filled into the donor chamber 4.

Over a period of 120 minutes, a photo of the respective filled Franz diffusion cells 1 was taken at regular intervals in order to document the osmosis process.

In the case of the compositions having osmotically active substance, the initial liquid level 7 in the acceptor chamber 3 sank due to osmosis-driven diffusion through the membrane 6. This can be read from a drop in the initial liquid level 7 in the indicator tube 8.

Without osmotically active substance in the non-inventive composition as the control, liquid passes from the upper donor chamber 4 into the lower acceptor chamber 3 and the liquid level rises.

This clearly shows that the tested composition having an osmotically and hygroscopically active substance, due to a concentration gradient, is capable of drawing liquid out of a physiological saline solution through a simulated sting channel.

Conversely, a substance without an osmotically active substance results in a reverse diffusion of liquid toward the physiological salt solution being observed.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1.-9. (canceled)

10. A composition for use in a treatment of skin irritations caused by plant or animal effects on or in a skin, the composition comprising an osmotically active substance, a hygroscopically active substance, or an osmotically active substance and a hygroscopically active substance.

11. The composition as claimed in claim 10, wherein the osmotically active substance is a salt.

12. The composition as claimed in claim 10, wherein the osmotically active substance is a sugar.

13. The composition as claimed in claim 10, wherein the hygroscopically active substance is a salt.

14. The composition as claimed in claim 10, wherein the hygroscopically active substance is a sugar.

15. The composition as claimed in claim 10, wherein the osmotically active substance, the hygroscopically active substance, or the osmotically active substance and the hygroscopically active substance is/are present at a concentration of 1% to 30% by weight of the composition.

16. The composition as claimed in claim 10, wherein the osmotically active substance, the hygroscopically active substance, or the osmotically active substance and the hygroscopically active substance is/are present at a concentration of 1.5% to 2.5% by weight of the composition.

17. The composition as claimed in claim 10, further comprising a solvent.

18. The composition as claimed in claim 17, wherein the solvent is water.

19. The composition as claimed in claim 10, further comprising a gel former.

20. The composition as claimed in claim 10, further comprising at least one additive.

21. The composition as claimed in claim 20, wherein the at least additive is selected from humectants, plant extracts, or buffers.

22. A method comprising:
applying the composition of claim 10 topically to a skin of a human or animal.

23. The method of claim 22, wherein the composition is in a form of a cream, an emulsion, or of a preparation for a stick, roll-on, patch or a spray.

24. The method of claim 22, wherein the composition is in a form of a gel.

25. The method of claim 22, wherein the composition is applied is an area of a skin irritation.

26. The method of claim 25, wherein the skin irritation is a sting or a bite by an arthropod.

27. The method of claim 25, wherein the skin irritation is a plant toxin.
